(19) United States
(12) Patent Application Publication
(10) Pub. No.: US 2016/0287248 A1
(43) Pub. Date: Oct. 6, 2016

(54) GEAR MECHANISM FOR ROTATING DRIVE SHAFT

(71) Applicants: Shalom Levin, Atlit (IL); Nir Altman, Kibbutz Kfar Etzion (IL)

(72) Inventors: Shalom Levin, Atlit (IL); Nir Altman, Kibbutz Kfar Etzion (IL)

(21) Appl. No.: 14/674,226

(22) Filed: Mar. 31, 2015

(51) Int. Cl. A61B 17/068; F16H 31/00; A61B 17/00; F16H 19/04

(52) U.S. Cl. CPC A61B 17/068; F16H 19/04; F16H 31/001; A61B 17/00234; F16H 2019/046; A61B 2017/0046; A61B 2017/00367

(57) ABSTRACT

A surgical device includes a housing and a trigger connected to the housing. The trigger is arranged to rotate a drive shaft by a gear mechanism. Linear movement of a linear movable element causes rotation of a rotatable gear operatively linked to the drive shaft.

### FIELD OF THE INVENTION

The present invention relates generally to surgical devices that have a rotating drive shaft, such as but not limited to, a tacker for applying surgical fasteners, and particularly to a gear mechanism for rotating the drive shaft.

### BACKGROUND OF THE INVENTION

A number of surgical devices employ a rotating drive shaft, which is used, for example, to deploy surgical devices, such as rotary tacks, in laparoscopic, endoscopic and other procedures, such as for hernia repairs and the like.

Tackers for applying such rotary tacks are well known. A typical tacker drive mechanism of the prior art is described in EP1908409. In this tacker, a rotatable gear is attached to a trigger so that squeezing the trigger rotates the rotatable gear. The rotatable gear meshes with a trigger gear. Squeezing the trigger rotates the rotatable gear, which causes rotation of the trigger gear, which in turn causes rotation of an idler gear which meshes with a gear train operatively linked to the drive shaft, thereby causing rotation of the drive shaft.

The tacker is designed so that rotation of the drive shaft causes deployment of a rotary tack or other coil fastener. It is important that the drive shaft rotates sufficiently to deploy the tack but must not over-rotate after deploying the tack; this prevents more than one coil fastener from being deployed upon a single pull of the trigger. In EP1908409, this is accomplished by stops protruding from the idler gear which can abut against blocking members in the handle housing. The stops limit the travel of the idler gear and thus prevent over-rotation of the drive shaft.

Another problem which must be solved in such tackers is preventing the trigger from returning to its initial position before the trigger has been fully squeezed. If the user were to release the trigger in the middle of deploying the coil fastener, the trigger would not be positioned to deploy the next fastener. In EP1908409, this problem is solved with a ratchet and pawl mechanism which engages one of gears and prevents that gear from rotating backwards. This prevents the trigger from returning to the initial position until the trigger has been fully depressed. The pawl of the ratchet and pawl mechanism is mounted on a pivotable leaf spring. This is because the position of the pawl relative to the gear can change, so the pawl must be biased towards the gear.

### SUMMARY OF THE INVENTION

The present invention seeks to provide an improved gear mechanism for rotating the drive shaft of a surgical device, such as but not limited to, tackers, endoscopic devices, laparoscopic devices and others, as is described more in detail hereinbelow.

In particular, without limitation, the present invention is distinguished from the prior art by at least three features. First, the trigger is operatively linked to the drive shaft by conversion of linear motion to rotational motion, such as by means of a gear rack meshing with a rotating (spur) gear. Second, prevention of more than one coil fastener from being deployed upon a single pull of the trigger is accomplished not by means of stops on one of the rotatable (idler) gears, but rather by limiting the movement of the linear movable element (e.g., gear rack). Third, the problem that exists in the prior art of preventing the trigger from returning to its initial position before the trigger has been fully squeezed does not exist in the present invention. In the present invention, squeezing the trigger causes a striking element to strike and rotate a rotatable gear that ultimately causes rotation of the drive shaft. There is no problem if the trigger is released before being fully squeezed, because the trigger is free to return to its starting position and the striking element will move to the striking position upon the next squeeze of the trigger. Furthermore, a type of one-way mechanism is provided that arrests movement of the rotatable gear (with a simple pawl) while at the same time the striking element is free to move back to the striking position and be ready for the next squeeze of the trigger. The striking element is a unique cantilevered striking element, as is explained hereinbelow.

There is thus provided in accordance with a non-limiting embodiment of the present invention a surgical device including a housing, and a trigger connected to the housing, the trigger arranged to rotate a drive shaft by a gear mechanism, and a linear movable element operatively connected to a rotatable gear which is operatively linked to the drive shaft, wherein linear movement of the linear movable element causes rotation of the rotatable gear to rotate the drive shaft.

In accordance with a non-limiting embodiment of the present invention the linear movable element includes a stop arranged to abut against a portion of the housing, the stop limiting the linear movement of the linear movable element.

In accordance with a non-limiting embodiment of the present invention the linear movable element includes a gear rack that meshes with the rotatable gear.

In accordance with a non-limiting embodiment of the present invention the rotatable gear is attached to a drive wheel and the drive wheel is arranged to rotate a drive gear, which is operatively linked to the drive shaft, in only one rotational direction.

In accordance with a non-limiting embodiment of the present invention the drive wheel has a striking element including a free end arranged to push against an inner protrusion formed on the drive gear. The striking element may be cantilevered and may have a fixed base opposite the free end. The striking element may taper in thickness from the fixed base to the free end. The protrusion may have a curved rear surface against which the free end can deflect.

In accordance with a non-limiting embodiment of the present invention a fixed pawl is attached to an inner portion of the housing and is in ratcheted engagement with the drive gear.

### BRIEF DESCRIPTION OF THE DRAWINGS

The present invention will be understood and appreciated more fully from the following detailed description taken in conjunction with the drawings in which:

FIG. 1 is a simplified pictorial illustration of a surgical device for rotating a drive shaft, constructed and operative in accordance with a non-limiting embodiment of the present invention;

FIG. 2 is a simplified pictorial illustration of a trigger and gear mechanism of the surgical device, constructed and operative in accordance with a non-limiting embodiment of the present invention;

FIGS. 3 and 4 are simplified pictorial and cutaway side-view illustrations, respectively, of a portion of the gear mechanism, showing a gear rack meshing with a spur gear of a drive wheel;

FIG. 5 is a simplified pictorial illustration of a drive gear assembled with the drive wheel that has the spur gear;

FIGS. 6A and 6B are simplified pictorial illustrations, taken at different views, of the drive wheel with the spur gear, wherein the drive wheel has a cantilevered striking element, in accordance with a non-limiting embodiment of the present invention; and

FIGS. 7A-7D are simplified front-view illustrations of the orientation of the drive wheel with respect to the drive gear during operation of the surgical device, in accordance with a non-limiting embodiment of the present invention.

### DETAILED DESCRIPTION OF EMBODIMENTS

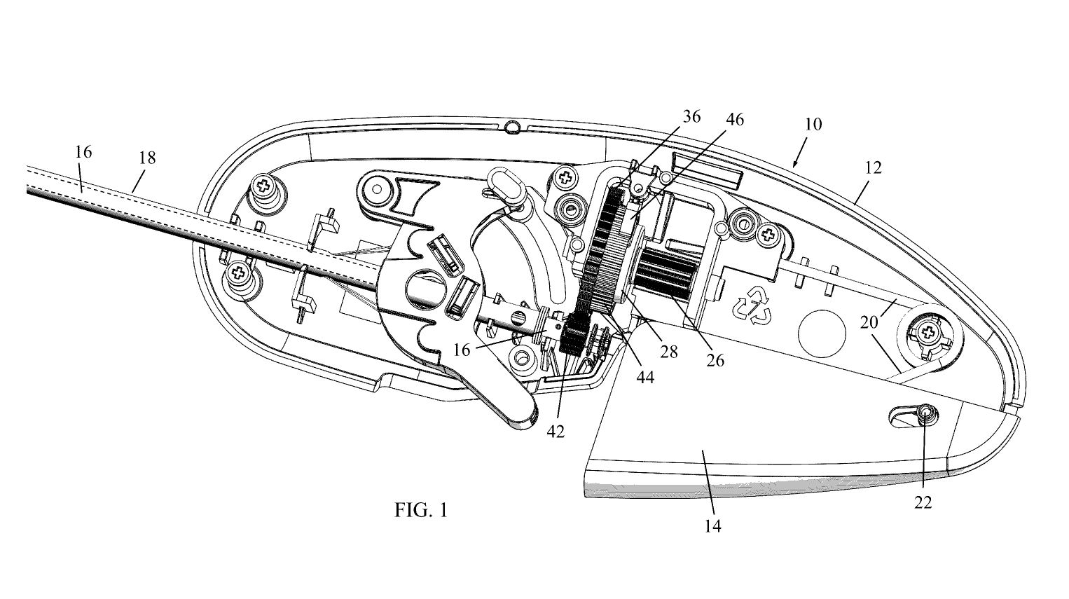

Reference is now made to FIG. 1, which illustrates surgical device 10 for rotating a drive shaft, constructed and operative in accordance with a non-limiting embodiment of the present invention.

A housing 12 (also called handle housing or handle) of surgical device 10 houses a deployment trigger 14 (referred to simply as trigger 14) for rotating a drive shaft 16 housed in an outer tube 18. Trigger 14 may be spring-loaded by a spring 20. Trigger 14 pivots about a pivot 22. Squeezing trigger 14 (upwards, clockwise in the sense of the drawing) causes rotation of the drive shaft 16 through a gear train, as is described hereinbelow. An applicator arm (not shown in FIG. 1) is connected to the drive shaft 16. In the case of a tacker, operation of trigger 14 causes the drive shaft 16 to rotate so as to distally advance coil fasteners (e.g., rotary tacks) from the applicator arm for deployment in tissue.

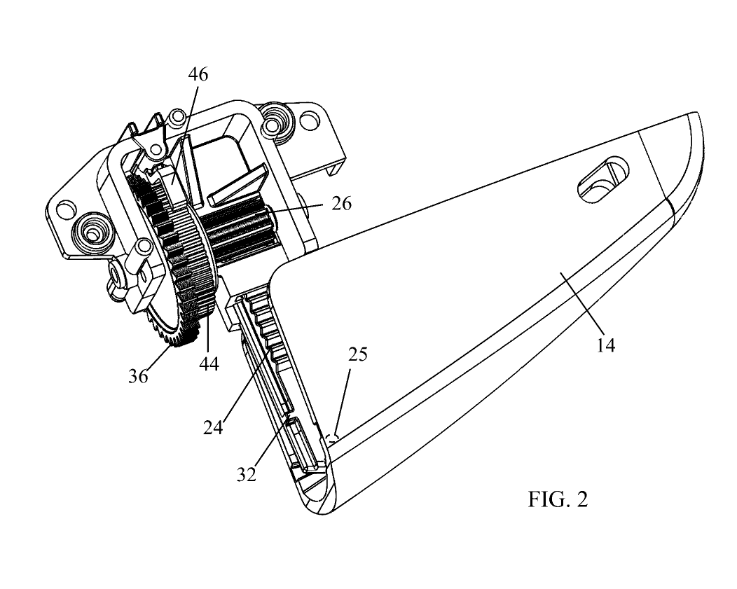

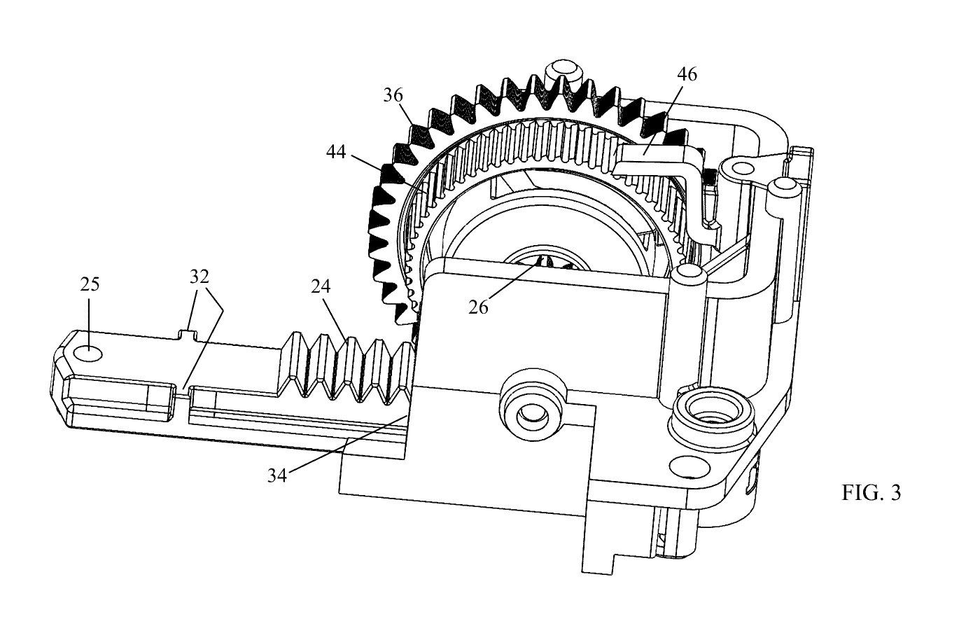

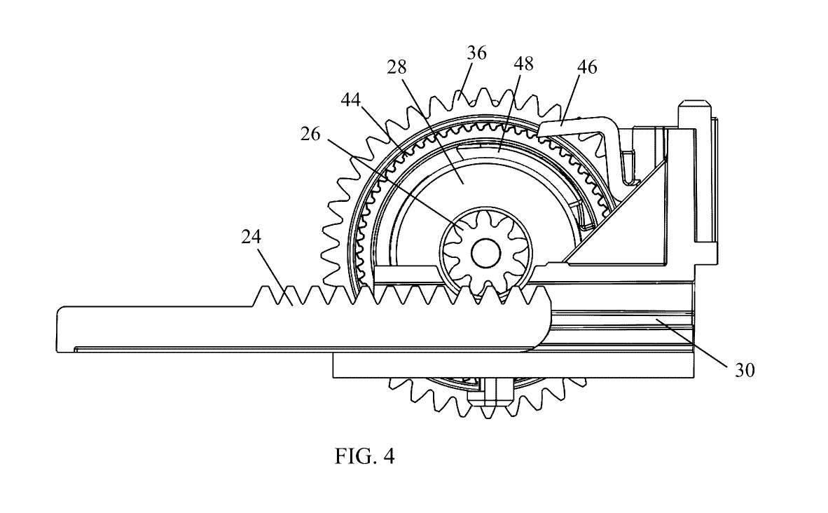

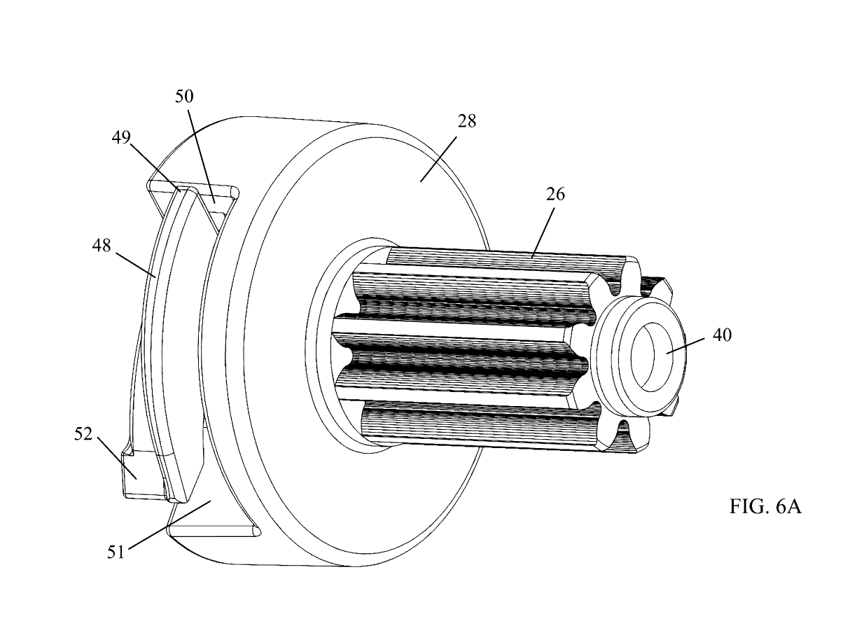

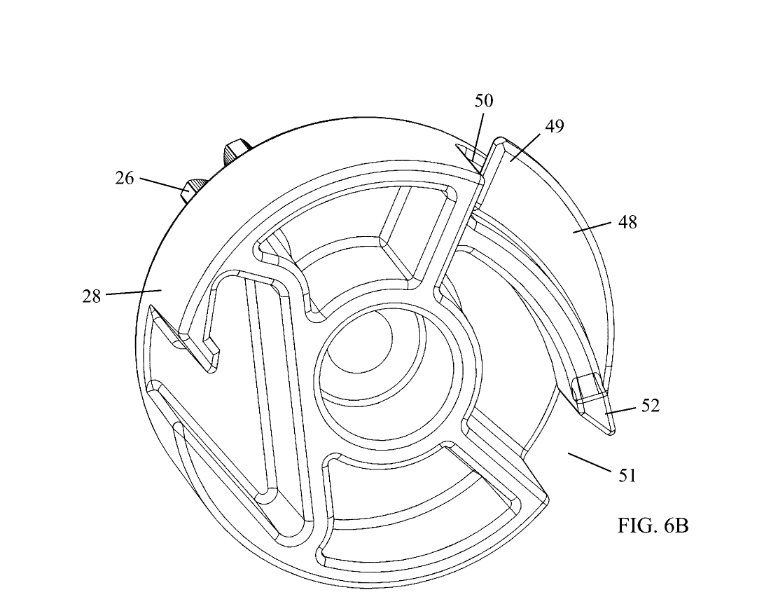

Reference is now made to FIGS. 2-4, which illustrate the gear train (gear mechanism) connected to trigger 14. The gear mechanism includes a linear movable element referred to as a gear rack 24, which meshes with a spur gear 26 of a drive wheel 28 (the construction of drive wheel 28 is described more in detail hereinbelow with references to FIGS. 6A and 6B). The gear rack 24 may be constrained to travel in a track 30 (FIG. 4). Although trigger 14 pivots about pivot 22 (FIG. 1), gear rack 24 moves linearly because gear rack 24 is pivoted to trigger 14 at a pivot 25 (e.g., a pin) (FIGS. 2 and 3). More specifically, in the illustrated embodiment, there is an oval cutout around the pin that serves as the pivot so that as trigger 14 is squeezed, gear rack 24 can move linearly in track 30 and can move freely about in the oval cutout so there is no interference with the linear movement in the track. Squeezing trigger 14 moves gear rack 24 linearly to the right in the sense of FIG. 4, thereby causing spur gear 26 to turn counterclockwise in the sense of FIG. 4. In this manner, trigger 14 is operatively linked to the drive shaft by conversion of linear motion to rotational motion. The mechanism is simpler, has less parts and is less costly than the prior art.

Gear rack 24 is provided with one or more stops 32 (FIGS. 2 and 3). The linear travel of gear rack 24 is limited by stop 32 abutting against a portion of housing 12, such as an abutment 34 (FIG. 3). This limitation on the movement of the linear movable element (gear rack 24) correspondingly limits the rotation of the spur gear 26 and drive wheel 28 and thus limits the rotation of the drive shaft 16. This prevents more than one coil fastener from being deployed upon a single pull of the trigger 14. The mechanism is simpler, has less parts and is less costly than the prior art. In addition, the stop or stops 32 on the linear movable element are more robust and accurate than the prior art.

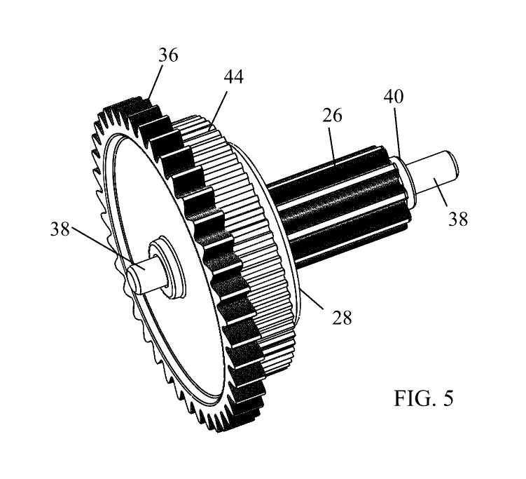

Reference is now made to FIGS. 1 and 5. The drive wheel 28 is assembled with a drive gear 36. Drive gear 36 is mounted on a shaft 38 (FIG. 5) that passes through a hollow shaft 40 (FIGS. 5 and 6A) of spur gear 26. As seen in FIG. 1, drive gear 36 meshes with a pinion gear 42 mounted at the end of drive shaft 16 to cause rotation of drive shaft 16. Drive gear 36 rotates together with a twin gear 44 attached thereto, generally of smaller diameter than gear 36. A fixed pawl 46 attached to an inner portion of housing 12 is in ratcheted engagement with twin gear 44. Pawl 46 permits twin gear 44 to rotate along with drive gear 36 in the same direction as spur gear 26. Pawl 46 prevents twin gear 44 and drive gear 36 from rotating in the direction opposite to spur gear 26.

The combination of the drive wheel 28, drive gear 36 and pawl 46 is a one-way mechanism; drive wheel 28 rotates drive gear 36 in the deployment direction but does not rotate drive gear 36 in the opposite direction, as is now explained with additional reference to FIGS. 6A and 6B.

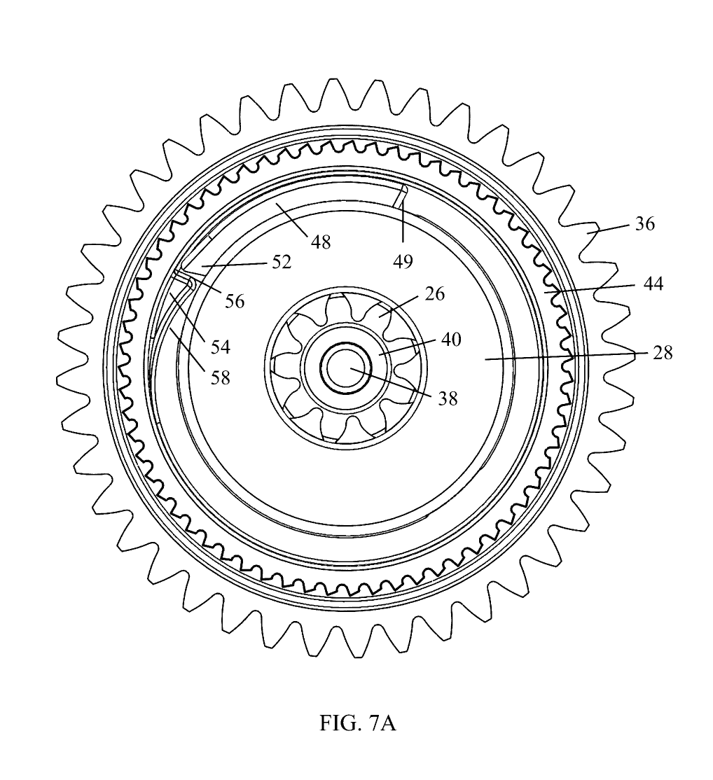

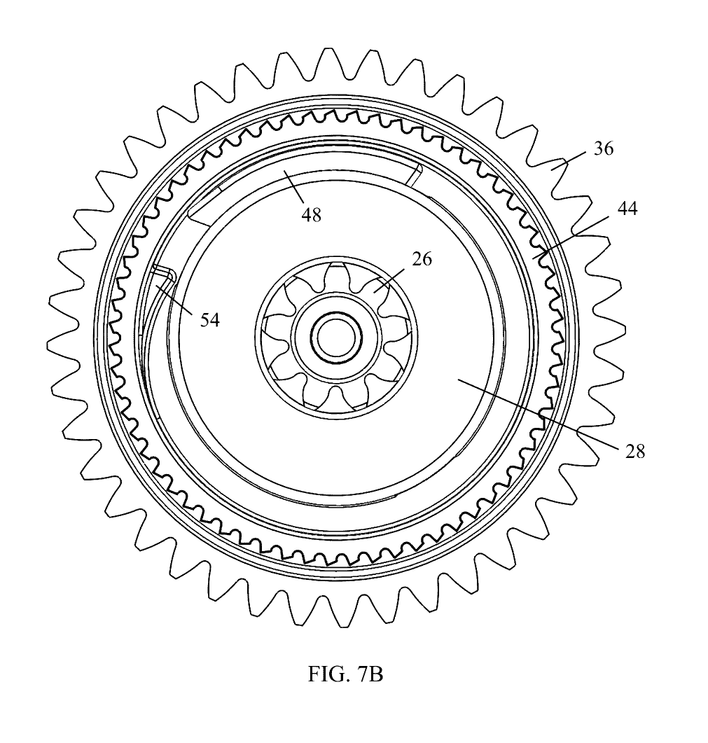

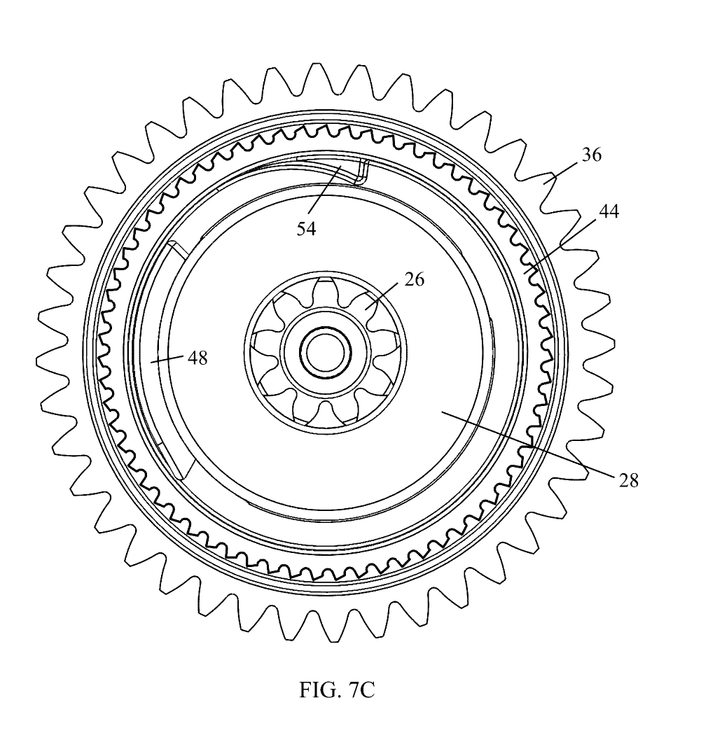

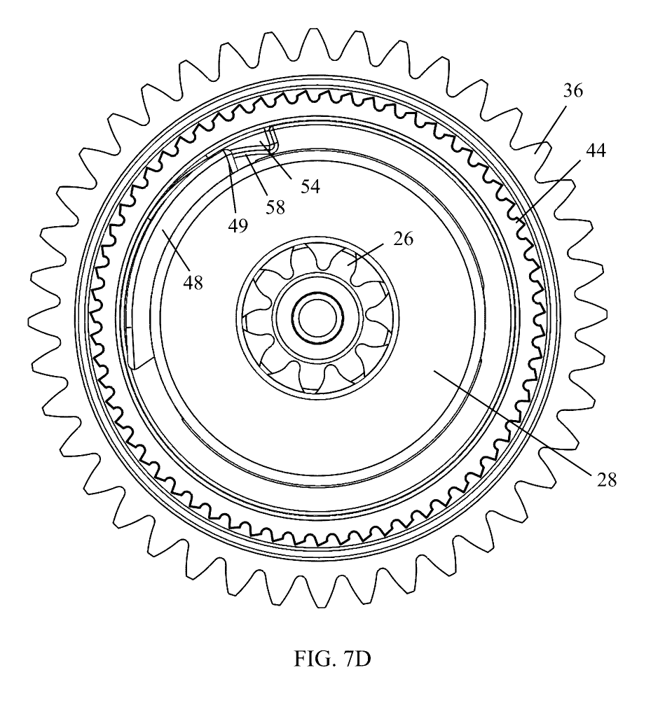

Drive wheel 28 has a striking element 48 which is cantilevered to the rest of drive wheel 28. As seen in FIGS. 6A and 6B, striking element 48 has a base 49 fixed to a wall 50 of a cutout portion 51 formed in drive wheel 28. Striking element 48 has a free end 52 opposite fixed base 49. Striking element 48 tapers in thickness from base 49 to free end 52. Since striking element 48 is cantilevered, free end 52 can bend radially inwards. Striking element 48 strikes (pushes) against a portion 54 of drive gear 36, such as an inner protrusion 54 (FIG. 7A) formed on drive gear 36 radially inwards of twin gear 44. Protrusion 54 has a flat face 56 against which the free end 52 of striking element 48 strikes (this is called the striking position). Protrusion 54 has a curved rear surface 58 against which striking element 48 deflects so that striking element 48 can ride over protrusion 54 and snap back to the striking position, as is now explained with reference to FIGS. 7A-7D, which illustrate drive wheel 28 with respect to drive gear 36 during operation of the surgical device.

FIG. 7A illustrates the striking position. The free end 52 of striking element 48 of drive wheel 28 strikes flat face 56 of protrusion 54 of drive gear 36. This is caused by squeezing the trigger to rotate the spur gear 26 and drive wheel 28 (via the gear rack) counterclockwise and thus rotate drive gear 36 counterclockwise (in the sense of FIG. 7A). The stop or stops arrest the movement of the gear rack and only one coil fastener is deployed.

In FIG. 7B, the trigger has been released. The backward movement of the trigger causes the gear rack to turn spur gear 26 and drive wheel 28 clockwise. Striking element 48 rotates clockwise away from protrusion 54 of drive gear 36. Pawl 46 (not shown here) holds drive gear 36 and twin gear 44 stationary. In other words, pawl 46 prevents twin gear 44 and drive gear 36 from rotating in the direction opposite to drive wheel 28.

In FIG. 7C, the trigger has been further released but not yet fully released. The backward movement of the trigger causes the gear rack to further turn spur gear 26 and drive wheel 28 clockwise. Striking element 48 rotates further clockwise away from protrusion 54 of drive gear 36, which is held stationary by pawl 46.

In FIG. 7D, the trigger has been further released but not yet fully released. The fixed base 49 of striking element 48 rides over the curved rear surface 58 of protrusion 54, thereby deflecting striking element 48 inwards. As the trigger is further released to the fully released position, striking element 48 completely rides over the curved rear surface 58 (in the clockwise direction) and snaps over protrusion 54 so that the free end 52 of striking element 48 returns to the striking position of FIG. 7A. The resiliency of the cantilevered striking element 48 permits such movement over the protrusion 54. The surgical device is now ready for the next round of deployment.

What is claimed is:

1. A surgical device comprising:
a housing;
a trigger connected to said housing, said trigger arranged to rotate a drive shaft by a gear mechanism; and
a linear movable element operatively connected to a rotatable gear which is operatively linked to said drive shaft, wherein linear movement of said linear movable element causes rotation of said rotatable gear to rotate said drive shaft.

2. The surgical device according to claim 1, wherein said linear movable element comprises a stop arranged to abut against a portion of said housing, said stop limiting the linear movement of said linear movable element.

3. The surgical device according to claim 1, wherein said linear movable element comprises a gear rack that meshes with said rotatable gear.

4. The surgical device according to claim 1, wherein said rotatable gear is attached to a drive wheel and said drive wheel is arranged to rotate a drive gear, which is operatively linked to said drive shaft, in only one rotational direction.

5. The surgical device according to claim 4, wherein said drive wheel has a striking element comprising a free end arranged to strike an inner protrusion formed on said drive gear.

6. The surgical device according to claim 5, wherein said striking element is cantilevered and has a fixed base opposite said free end.

7. The surgical device according to claim 6, wherein said striking element tapers in thickness from said fixed base to said free end.

8. The surgical device according to claim 6, wherein said protrusion has a curved rear surface against which said free end can deflect.

9. The surgical device according to claim 4, wherein said drive gear is mounted on a shaft that passes through a hollow shaft of said rotatable gear.

10. The surgical device according to claim 4, wherein said drive gear meshes with a pinion gear mounted on said drive shaft to cause rotation of said drive shaft.

11. The surgical device according to claim 4, wherein a fixed pawl attached to an inner portion of said housing is in ratcheted engagement with said drive gear.

12. A surgical device comprising:
a housing; and
a trigger connected to said housing, said trigger arranged to rotate a drive shaft by a gear mechanism;
wherein said gear mechanism comprises a rotatable gear attached to a drive wheel that has a striking element arranged to be in a striking position to strike a portion of said drive gear to cause rotation of said drive gear and wherein said drive gear is operatively linked to rotate said drive shaft, and further comprising a one-way mechanism capable of arresting movement of said drive gear while simultaneously permitting said striking element to move to the striking position.

13. The surgical device according to claim 12, wherein said one-way mechanism comprises a fixed pawl attached to an inner portion of said housing and in ratcheted engagement with said drive gear.

14. The surgical device according to claim 12, wherein said striking element is cantilevered and has a fixed base opposite a free end.

15. The surgical device according to claim 14, wherein said protrusion has a curved rear surface against which said free end can deflect so that said striking element is arranged to ride over said protrusion to return to the striking position.
